(19) United States
(12) Patent Application Publication
(10) Pub. No.: US 2024/0266004 A1
(43) Pub. Date: Aug. 8, 2024

(54) DENOISING DIFFUSION MODEL FOR COARSE-GRAINED MOLECULAR DYNAMICS

(71) Applicant: Microsoft Technology Licensing, LLC, Redmond, WA (US)

(72) Inventors: Rianne VAN DEN BERG, Amsterdam (NL); Victor GARCÍA SATORRAS, Amsterdam (NL); Chin-Wei HUANG, Amsterdam (NL); Daniel ZUEGNER, Berlin (DE); Frank NOE, Berlin (DE); Cecilia CLEMENTI, Berlin (DE); Robert PINSLER, Cambridge (GB); Marloes Elisabeth ARTS, Copenhagen (DK); Marco FEDERICI, Diemen (NL)

(73) Assignee: Microsoft Technology Licensing, LLC, Redmond, WA (US)

(21) Appl. No.: 18/314,630

(22) Filed: May 9, 2023

Related U.S. Application Data
(60) Provisional application No. 63/481,557, filed on Jan. 25, 2023.

(51) Int. Cl. G16C 10/00

(52) U.S. Cl. CPC G16C 10/00

(57) ABSTRACT

A computing system including a processor configured to receive atomistic molecular dynamics simulation data of training-time conformers of an atomistic structure of a molecule. The processor is further configured to compute coarse-grained molecular dynamics simulation data based at least in part on the atomistic molecular dynamics simulation data. The coarse-grained molecular dynamics simulation data is computed at least in part by converting the atomistic structure into a coarse-grained structure. The processor is further configured to train a denoising diffusion model using the coarse-grained molecular dynamics simulation data. At the denoising diffusion model, the processor is further configured to receive a runtime conformer of the coarse-grained structure and generate a coarse-grained force field estimate associated with the runtime conformer. The processor is further configured to output the coarse-grained force field estimate to a molecular dynamics simulation module and generate a molecular dynamics simulation based on the coarse-grained force field estimate.

### CROSS REFERENCE TO RELATED APPLICATIONS

This application claims priority to U.S. Provisional Patent Application Ser. No. 63/481,557, filed Jan. 25, 2023, the entirety of which is hereby incorporated herein by reference for all purposes.

### BACKGROUND

Coarse-grained (CG) molecular dynamics (MD) promises to scale simulations of molecules to larger spatial and time scales than currently accessible through atomistic MD simulations. Scaling up MD by orders of magnitude would enable new studies on macromolecular dynamics over longer ranges of time, such as large protein folding events and slow interactions between large molecules. CG molecular dynamics enables the study of biological processes at temporal and spatial scales that would be intractable at an atomistic resolution. For example, CG molecular dynamics simulations are sometimes used in pharmaceutical research.

To obtain a CG simulation model, one first maps the all-atom, or fine-grained, representation to a coarse-grained representation, for example, by grouping certain atoms together to form CG beads. Second, a CG force field is computed. By using the CG force field that acts on the CG beads, CG molecular dynamics simulations reproduce relevant features of molecular systems. In top-down approaches, a CG model is defined to reproduce ensemble averages of specific observables as experimentally measured and/or simulated on fine-grained models. In bottom-up approaches, a CG model reproduces the behavior (e.g., thermodynamics or kinetics) of a fine-grained model. In the latter case, one previous approach is to define a CG force field for the chosen CG representation by enforcing thermodynamic consistency. When this approach is used, simulations following the CG model have approximately the same equilibrium distribution as the equilibrium distribution obtained by projecting equilibrated all-atom simulations onto the CG resolution.

### SUMMARY

According to one aspect of the present disclosure, a computing system is provided, including a processor configured to receive atomistic molecular dynamics simulation data of a plurality of training-time conformers of an atomistic structure of a molecule. The processor is further configured to compute coarse-grained molecular dynamics simulation data of the molecule based at least in part on the atomistic molecular dynamics simulation data. The coarse-grained molecular dynamics simulation data is computed at least in part by converting the atomistic structure into a coarse-grained structure of the molecule. The processor is further configured to train a denoising diffusion model using the coarse-grained molecular dynamics simulation data. At the denoising diffusion model, the processor is further configured to receive a runtime conformer of the coarse-grained structure. The processor is further configured to generate a coarse-grained force field estimate associated with the runtime conformer. The processor is further configured to output the coarse-grained force field estimate to a molecular dynamics simulation module. The processor is further configured to generate a molecular dynamics simulation of the molecule at the molecular dynamics simulation module based at least in part on the coarse-grained force field estimate.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 schematically shows a computing system when training data for a denoising diffusion model is generated, according to one example embodiment.

FIG. 2 shows examples of atomistic structures and coarse-grained structures of alanine dipeptide and of fast-folding proteins, according to the example of FIG. 1.

FIG. 3 schematically shows the computing system during training of the denoising diffusion model, according to the example of FIG. 1.

FIG. 4 schematically shows the computing system during runtime when the processor is configured to generate a coarse-grained force field estimate at the denoising diffusion model, according to the example of FIG. 1.

FIG. 5 schematically shows the computing system in an example in which the processor is further configured to generate a plurality of independent identically distributed samples at the denoising diffusion model, according to the example of FIG. 1.

FIG. 6 shows a plot of Jensen-Shannon divergence between joint distributions of dihedral angles in the coarse-grained representation of alanine dipeptide when the coarse-grained representation is modeled using different techniques, according to the example of FIG. 1.

FIG. 7A shows a flowchart of method for use with a computing system to perform training and inferencing at a denoising diffusion model used in molecular dynamics simulation, according to the example of FIG. 1.

FIG. 7B shows additional steps of the method of FIG. 7A that may be performed in some examples when training the denoising diffusion model.

FIG. 7C shows additional steps of the method of FIG. 7A that may be performed when the coarse-grained molecular dynamics simulation data is computed.

FIG. 7D shows additional steps of the method of FIG. 7A that may be performed to generate independent identically distributed samples from an equilibrium distribution of the coarse-grained structure.

FIG. 8 shows a schematic view of an example computing environment at which the computing system of FIG. 1 may be instantiated.

### DETAILED DESCRIPTION

Traditional bottom-up coarse-graining techniques that rely on the thermodynamic consistency principle have produced significant results in the last decade, in particular when used in combination with machine learning methods. Two of these bottom-up coarse-graining techniques are variational force matching and relative entropy minimization, as discussed in further detail below. Variational force matching minimizes the mean squared error between the model's CG forces and the atomistic forces projected onto the CG space. However, due to the stochastic nature of the projected forces, this noisy force-matching estimator has a large variance, leading to data-inefficient training. Variational force matching methods also require storing values of the atomistic forces.

Alternatively, relative entropy minimization approaches perform density estimation in the CG space without accessing atomistic forces. Relative entropy minimization methods are frequently equivalent to energy-based models. Since training these models requires drawing samples from the model iteratively to estimate log-likelihood gradients, relative entropy minimization methods demand significantly higher computational costs than variational force matching methods.

Flow-matching methods have also been recently introduced in order to alleviate the downsides of variational force matching methods and relative entropy minimization methods, such as sample inefficiency and the requirement to store values of atomistic forces. In a first density matching stage performed during flow matching, a CG density is modeled with an augmented normalizing flow. In a second learning stage with a force-matching-like objective, a deterministic CG force field is extracted. The deterministic CG force field may then be used in CG molecular dynamics simulations. Flow-matching improves performance on some fast-folding proteins. However, the learned CG models are sometimes quantitatively inaccurate in reproducing the thermodynamics of the corresponding fine-grained models, and scaling to larger proteins leads to instabilities.

In the systems and methods discussed below, denoising diffusion is used to train a generative model on equilibrium samples of CG structures. Training the generative model with a denoising loss function and a conservative score function yields a single model that may be used both to produce independent identically distributed (i.i.d.) CG samples and to estimate a CG force field for CG molecular dynamics simulations. The denoising diffusion model may be trained in a single-stage training setup that is simpler than the training setups used in other machine-learning-based CG molecular simulation approaches. In addition, the denoising diffusion model may have improved performance across several small- to medium-sized protein simulations, reproducing the CG equilibrium distribution and preserving dynamics of all-atom simulations such as protein folding events. The denoising diffusion model also allows scaling to larger protein sizes than flow-matching approaches.

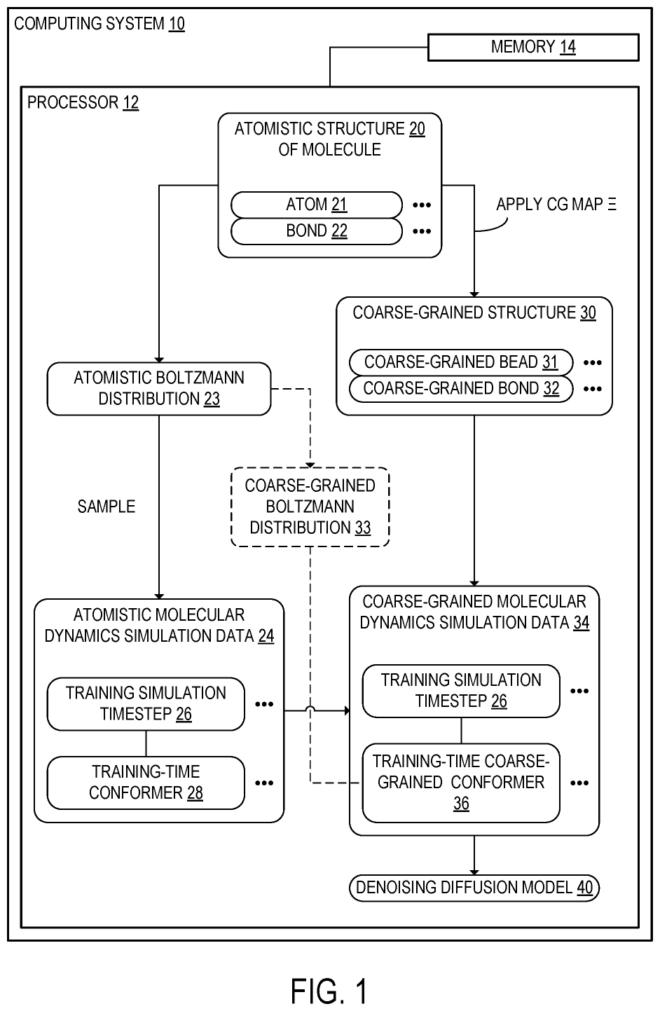

FIG. 1 schematically shows a computing system 10 when training data for a denoising diffusion model 40 is generated. The computing system 10 shown in FIG. 1 includes a processor 12 and memory 14. The processor 12 includes one or more processing devices such as one or more central processing units (CPUs), graphics processing units (GPUs), specialized hardware accelerators, or other processing devices. The memory 14 includes one or more memory devices, which may be volatile storage devices and/or non-volatile storage devices.

In some examples, components of the computing system 10 may be provided within a single computing device. Alternatively, the components of the computing system 10 may be distributed among a plurality of physical computing devices, such as a plurality of server computing devices located in a data center. In examples in which the computing system 10 includes a server computing device, that server device may be configured to communicate with a client computing device, such as to receive user input and to output results of requested computations to the user of the client computing device.

The denoising diffusion model 40, as shown in FIG. 1, is trained for use in simulating a molecule. The molecule may be defined using an atomistic structure 20 that indicates a plurality of atoms 21 included in the molecule. The atomistic structure 20 further specifies a plurality of bonds 22 between the atoms 21. Thus, the atomistic structure 20 may be a graph representation of the molecule.

The processor 12 is further configured to receive atomistic molecular dynamics simulation data 24. The atomistic molecular dynamics simulation data 24 is generated by simulating a plurality of training-time conformers 28 of the atomistic structure 20 of the molecule. In the atomistic molecular dynamics simulation data 24, the training-time conformers are associated with a respective plurality of training simulation timesteps 26. In some examples, as shown in FIG. 1, the processor 12 is configured to sampling the plurality of training-time conformers 28 from an atomistic Boltzmann distribution 23 of the atomistic structure 20, as discussed in further detail below.

The processor 12 is further configured to convert the atomistic structure 20 into a CG structure 30 of the molecule. The processor 12 is configured to generate the CG structure 30 at least in part by reducing a dimensionality of the atomistic structure 20. The CG structure 30 includes a plurality of CG beads 31 that are coupled by a plurality of CG bonds 32, with each of the CG beads 31 including one or more of the atoms 21 included in the atomistic structure 20. Accordingly, when the CG structure 30 is generated, groups of the atoms 21 are represented in a combined manner as the CG beads 31.

Computation of the CG structure 30 may be described with a coarse-graining map Ξ: [symbol] ^{3N}→ [symbol] ³ⁿ that transforms a high-dimensional atomistic representation x∈ [symbol] ^{3N} to a lower-dimensional CG representation z∈ [symbol] ³ⁿ, where n<<N. For molecular systems, the CG map Ξ is usually linear, such that Ξ∈ [symbol] ^{3N×3n}, and returns the Cartesian coordinates z of the CG beads 31 as a linear combination of the Cartesian coordinates x of a set of representative atoms 21.

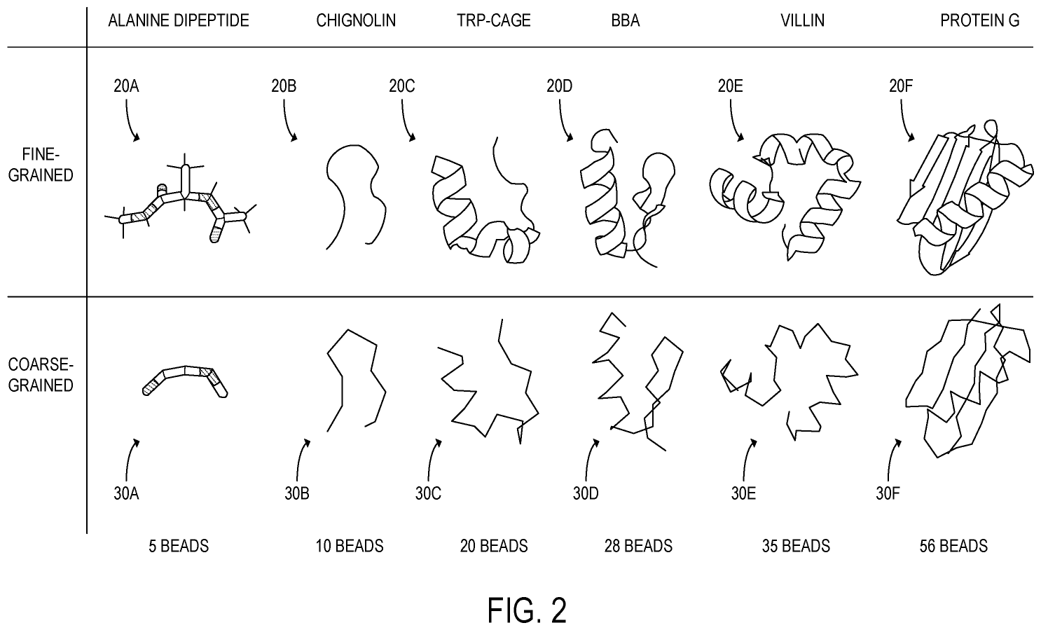

FIG. 2 shows examples of atomistic structures 20 (also referred to as fine-grained structures) and CG structures 30 for alanine dipeptide and for the fast-folding proteins chignolin, Trp-cage, Bba, villin, and protein G. In the example of FIG. 2, the CG structure 30A generated from the atomistic structure 20A of alanine dipeptide includes five CG beads, which are represented as nodes of a chain structure in the example of FIG. 2. The CG structure 30B generated from the atomistic structure 20B of chignolin includes 10 CG beads; the CG structure 30C generated from the atomistic structure 20C of Trp-cage includes 20 CG beads; the CG structure 30D generated from the atomistic structure 20D of Bba includes 28 CG beads; the CG structure 30E generated from the atomistic structure 20E of villin includes 35 CG beads; and the CG structure 30F generated from the atomistic structure 20F of protein G includes 56 CG beads. These proteins may be coarse-grained by slicing out the C_{α} atom for every amino acid, giving rise to one bead per residue.

Returning to the example of FIG. 1, the processor 12 is further configured to compute CG molecular dynamics simulation data 34 of the molecule based at least in part on the atomistic molecular dynamics simulation data 24 and the CG structure 30. The CG molecular dynamic simulation data 34 may include a plurality of training-time CG conformers 36 respectively associated with the plurality of training simulation timesteps 26 used in the atomistic molecular dynamics simulation data 24. Computing the CG molecular dynamics simulation data 34 may include mapping the plurality of training-time conformers 28 onto the CG structure 30 to obtain a plurality of training-time CG conformers 36 included in the CG molecular dynamics simulation data 34. Accordingly, of the training-time CG conformers 36 may be samples from a CG Boltzmann distribution 33.

The processor 12 is further configured to train the denoising diffusion model 40 using the CG molecular dynamics simulation data 34. Converting the atomistic molecular dynamics simulation data 24 into the CG molecular dynamics simulation data 34 prior to training the denoising diffusion model 40 may increase the efficiency of the training by compressing the atomistic molecular dynamics simulation data 24 into a more quickly learnable form.

Computation of force field data at the denoising diffusion model 40 is discussed below. As mentioned above, the atomistic molecular dynamics simulation data 24 may be sampled from an atomistic Boltzmann distribution 23. The probability density of the atomistic structure 20 at a particular temperature [symbol] is described by the Boltzmann distribution q(x)∝ [symbol] , where U(x) is the potential energy of the system and k_{B} is the Boltzmann constant.

In some examples, the processor 12 may be further configured to compute a CG Boltzmann distribution 33 based at least in part on the atomistic Boltzmann distribution 23. By identifying the ensemble of atomistic configurations x that maps onto the CG configuration z, the probability density of CG configurations z may be explicitly expressed as the following CG Boltzmann distribution 33:

$$q ( z ) = \frac{\int δ ( Ξ ( x - z ) ) d x}{\int {e}^{- U ( x ′ ) / {k}_{B} 𝒥} {dx}^{′}}$$

where δ(·) is the Dirac delta function. Up to an additive constant, the above distribution uniquely defines the thermodynamically consistent effective CG potential of a mean force V(z). The mean force V(z) is given as follows:

$$V ( z ) = - {k}_{B} log q ( z ) + c s t . = - {k}_{B} log \int {e}^{- \frac{U ( x )}{}} δ ( Ξ ( x - z ) ) dx + cst .$$

In relevant molecular systems, the integral in the above equation is intractable. Therefore, methods that approximate thermodynamically consistent effective CG potentials have been proposed. Variational force matching is one existing method of approximating effective CG potentials. Under certain constraints applied to the CG map Ξ, a more tractable consistency equation between a coarse-grained force field −∇_{z}V(z) and an atomistic force field −∇ₓU(x) may be obtained. When the CG map Ξ is a linear map and when each CG bead 31 has at least one atom with a nonzero coefficient only for that specific CG head 31, the following relationship holds:

$$- {∇}_{z} V ( z ) = {𝔼}_{q ( X \vert Z )} [ {Ξ}_{f} ( - {∇}_{x} U ( x ) ) ]$$

where Ξ_{f} is a linear map whose coefficients are related to the linear coefficients of the CG map Ξ. The above equation for the CG force field may be used to approximate a thermodynamically consistent CG potential V_{θ}(z), expressed as a functional form with a set of parameters θ, by finding the values of the parameters that minimize the following variational loss:

$${𝔼}_{q ( x , z )} [ {‖ {∇}_{z} {V}_{θ} ( z ) - {Ξ}_{f} ( {∇}_{x} U ( x ) ) ‖}_{2}^{2} ]$$

Another existing approach to obtaining the CG forces is via relative entropy minimization, where optimizing the density implicitly leads to optimized mean potential functions. Concretely, the CG density is estimated by minimizing the relative entropy, which is expressed as a Kullback-Leibler divergence [symbol] _{q(z)}[log q(z)−log p_{θ}(z)], where p_{θ}(z) is a learned CG density function. Solving the above minimization problem is equivalent to optimizing the maximum likelihood when a finite number of samples is drawn from p(z). The approximate mean forces may be extracted from the optimized model density p_{θ}(z) using the relationship −∇_{z}V_{θ}(z)∝∇_{z} log p_{θ}(z). Unlike variational force matching, relative entropy minimization does not impose any constraints on the CG map Ξ, and no atomistic forces are required for training.

Typically, an unnormalized version of the learned CG density function p_{θ} is modeled by directly parameterizing the mean potential V_{θ}, yielding the relationship p_{θ}(z)∝ [symbol] . To minimize the relative entropy, the free energy (the normalizing constant) of the model may be estimated. Alternatively, i.i.d. samples may be drawn from the model and used to perform gradient estimation. However, both of these approaches are impractical for high-dimensional problems.

As an alternative to variational force matching and relative entropy maximization, explicit density models in the form of normalizing flows have instead been used in some existing CG force computation approaches. In principle, a standard normalizing flow has a tractable normalized density p_{θ}(z), thereby allowing for straightforward maximum-likelihood density estimation and force field learning. However, expressive invertible functions are difficult to learn. Instead, auxiliary normalizing flows may be used to model flow density. The introduction of auxiliary random variables increases the expressivity of the flow, at the cost of an intractable marginal likelihood, yielding a minimization objective that is a variational upper bound to the relative entropy. In addition, only a stochastic estimate of the CG force may be extracted from the auxiliary normalizing flow model. In order to distill a deterministic approximate mean force to simulate the CG dynamics, a teacher-student setup may be used. This two-stage approach is referred to as flow-matching.

As discussed above, a denoising diffusion model 40 for coarse-grained molecular dynamics is provided herein as an alternative to the conventional CG force modeling techniques discussed above. Denoising diffusion probabilistic models (DDPMs) sample from a probability distribution by approximating the inverse of a diffusion process: a denoising process. The diffusion (forward) process is defined as a Markov chain of L steps q(z_{1:L}|z₀)=Πᵢ₌₁^{L}q(zᵢ|zᵢ₋₁), where z₀ is a sample from the unknown data distribution q(z₀). The learned reverse process is defined as a reverse-time Markov chain of L denoising steps starting from the prior p(z_{L}). The denoising steps are expressed as p(z_{0:L}):=p(z_{L})Πᵢ₌₁^{L}p_{θ}(zᵢ₋₁|zᵢ).

For real-valued random variables, the distribution for the forward process may be Gaussian, such that q(zᵢ|zᵢ₋₁)= [symbol] (zᵢ; \sqrt{1−βᵢ}zᵢ₋₁, βᵢI). In this equation, {βᵢ} are fixed variance parameters that increase as a function of i such that the Markov chain has a standard normal stationary distribution. The reverse process distributions may have the same functional form:

$$p ( {z}_{L} ) = 𝒩 ( 0 , I )$$

$${p}_{θ} ( {z}_{i - 1} \vert {z}_{i} ) = 𝒩 ( {z}_{i - 1} , {μ}_{θ} ( {z}_{i} , i ) , {σ}_{i}^{2} I )$$

In the above equations, μ_{θ}(zᵢ, i) is the mean of the Gaussian distribution, expressed as a learnable function with parameters θ. σᵢ² is a fixed variance.

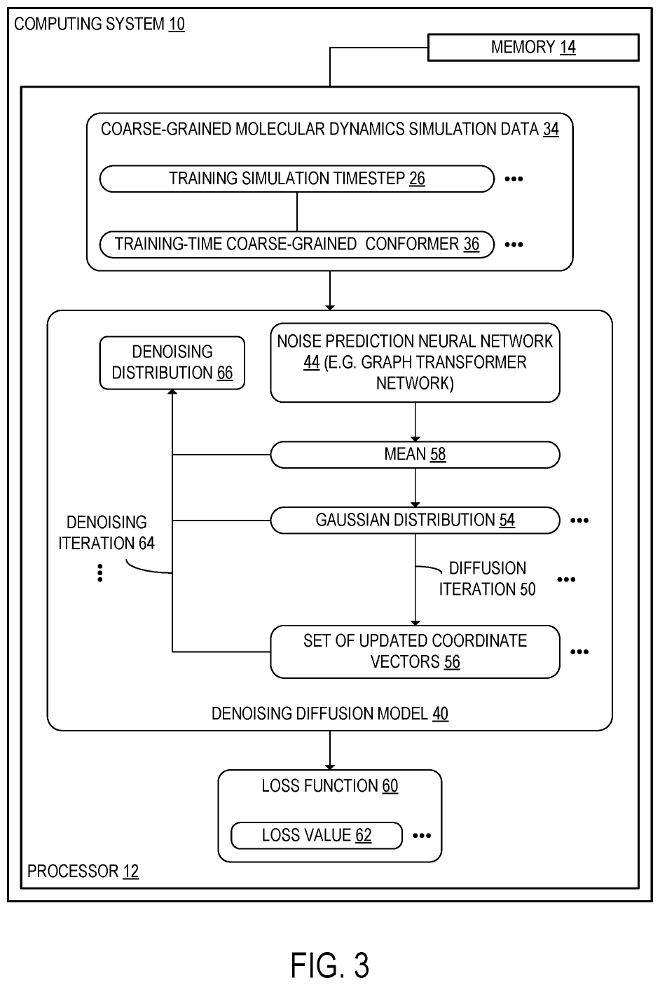

FIG. 3 schematically shows the computing system 10 in additional detail during training of the denoising diffusion model 40. In the example of FIG. 3, during a plurality of diffusion iterations 50 performed when training the denoising diffusion model, the processor 12 is configured to compute a respective plurality of sets of updated coordinate vectors 56 associated with the CG beads 31. The processor 12 may be configured to perform L diffusion iterations 50 starting from a training-time CG conformer 36. In addition, the processor 12 may be configured to perform each of the diffusion iterations 50 at least in part by sampling the set of updated coordinate vectors 56 from a respective Gaussian distribution 54. The Gaussian distribution 54 for the ith diffusion iteration 50 may be computed using the equation for q(zᵢ|zᵢ₋₁) discussed above. The set of updated coordinate vectors 56 computed at each of the diffusion iterations 50 includes the coordinates of the CG beads 31 after diffusion has been applied to the CG structure 30. When training is performed, closed-form marginalizations of the Gaussian distributions 54 may be used.

In addition, during each of the diffusion iterations 50, the processor 12 may be further configured to compute a mean 58 of the Gaussian distribution 54 at least in part by executing a noise prediction neural network 44. The mean 58 of the Gaussian distribution 54 for each diffusion iteration 50 may be parameterized as follows:

$${μ}_{θ} ( {z}_{i} , i ) = \frac{1}{\sqrt{{α}_{i}}} ( {z}_{i} - \frac{{β}_{i}}{\sqrt{1 - {\overline{α}}_{i}}} {ϵ}_{θ} ( {z}_{i} , i ) )$$

where ϵ_{θ}(zᵢ, i) is the noise prediction neural network 44. In the above equation, αᵢ=1−βᵢ and α̅ᵢ=Πₛ₌₁ⁱαₛ. The noise prediction neural network 44 may, for example, be a graph transformer network.

During training of the denoising diffusion model 40, subsequently to each of the diffusion iterations 50, the processor 12 may be further configured to perform a denoising iteration 64. In each of the denoising iterations 64, the processor 12 may be configured to compute a respective value of a denoising distribution 66 (indicated above as p(z_{0:L})). Accordingly, the denoising diffusion model 40 is configured to learn an inverse of the diffusion process.

The processor 12 is further configured to compute loss values 62 at a loss function 60 when training the denoising diffusion model 40. The mean 58 may be used to parameterize the Gaussian distribution included in the loss function 62. The loss function 60 used during training is given by:

$${\sum }_{i = 1}^{L} {K}_{i} {𝔼}_{q ( {z}_{0} )} {𝔼}_{𝒩 ( ϵ ; 0 , I )} [ {‖ ϵ - {ϵ}_{θ} ( \sqrt{{\overline{α}}_{i}} {z}_{0} + \sqrt{1 - {\overline{α}}_{i}} ϵ , i ) ‖}^{2} ]$$

In the above expression for the loss function 60, q(z₀) is the data distribution. Up to a constant, the above expression for the loss function 60 is a negative evidence lower bound when

$${K}_{i} = \frac{{β}_{i}^{2}}{2 {σ}_{i}^{2} {α}_{i} ( 1 - {\overline{α}}_{i} )}$$

In some examples, a reweighted loss with Kᵢ=1 may be used instead. The DDPM loss with Kᵢ=1 is equivalent to the following weighted sum of denoising score matching objectives:

$${\sum }_{i = 1}^{L} ( 1 - {\overline{α}}_{i} ) {𝔼}_{q ( {z}_{0} )} {𝔼}_{q ( {z}_{i} \vert {z}_{0} )} [ {‖ {s}_{θ} ( {z}_{i} , i ) - {∇}_{{z}_{i}} log q ( {z}_{i} \vert {z}_{0} ) ‖}^{2} ]$$

In the above expression for the sum of denoising score matching objectives, q(zᵢ|z₀)= [symbol] (zᵢ; \sqrt{α̅}z₀, (1−α̅ᵢ)I), and s_{θ}(zᵢ, i) represents a score model. The equivalence of the loss function 60 to the weighted sum of the denoising score matching objectives is achieved by relating the score model s_{θ}(zᵢ, i) to the noise prediction network ϵ_{θ}(zᵢ, i) through

$${s}_{θ} ( {z}_{i} , i ) = - \frac{{ϵ}_{θ} ( {z}_{i} , i )}{\sqrt{1 - {\overline{α}}_{i}}}$$

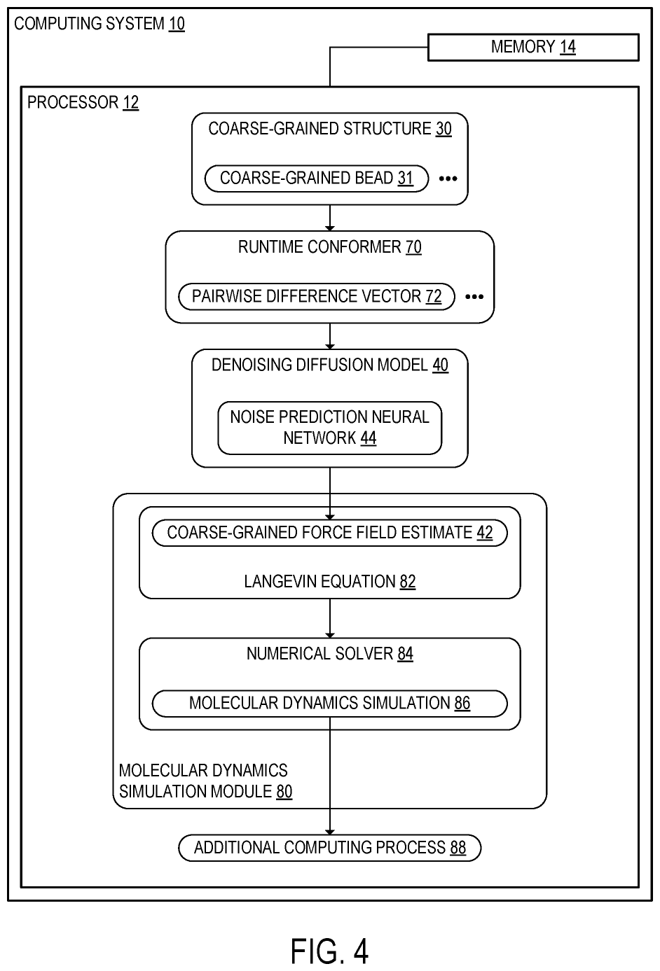

FIG. 4 schematically shows the computing system 10 during runtime when the processor 12 is configured to generate a CG force field estimate 42 at the denoising diffusion model 40. The CG force field estimate 42 is generated subsequently to receiving a runtime conformer 70 of the CG structure 30. In addition, according to the example of FIG. 4, the processor 12 is further configured to output the CG force field estimate 42 to a molecular dynamics simulation module 80.

At the molecular dynamics simulation module 80, the processor 12 is further configured to generate a molecular dynamics simulation 86 of the molecule based at least in part on the CG force field estimate 42. The molecular dynamics simulation 86 may be generated at least in part by executing a numerical solver 84 included in the molecular dynamics simulation module 80. The molecular dynamics simulation 86 may be a simulation of the movement of the CG beads 31 over time. The processor 12 may be further configured to output the molecular dynamics simulation 86 to an additional computing process 88, such as a graphical user interface (GUI) generating process or a biological interaction simulation program.

Given a sufficiently expressive model and a sufficient amount of data, the optimal score s_{θ*}(zᵢ, i) approximately matches the score ∇zᵢ log q(zᵢ), where q(zᵢ)=∫dz₀q(zᵢ|z₀)q(z₀) is the marginal distribution at level i of the forward diffusion process. At sufficiently low noise levels, the marginal distribution q(zᵢ) resembles the data distribution q(z₀), such that s_{θ*}(zᵢ, i) effectively approximates the score of the unknown data distribution. When the unknown data distribution is equal to the CG Boltzmann distribution 33, the optimal score s_{θ*}(zᵢ, i) at level i=1 therefore approximately matches the CG forces:

$${∇}_{z} log p ( z ) = \frac{- {∇}_{z} V ( z )}{} = \frac{{F}_{z}}{}$$

Using the relation between the score model s_{θ}(zᵢ, i) and the noise prediction network ϵ_{θ}(zᵢ, i), the approximate CG forces may be extracted from the denoising diffusion model as follows:

$${F}_{z}^{DFF} = - \frac{{k}_{B}}{\sqrt{1 - {\overline{α}}_{i}}} {ϵ}_{θ} * ( z , i )$$

This CG force field estimate 42 is referred to as the denoising force field.

Although, in principle, the lowest level (i=1) is expected to provide the most accurate CG force field estimate 42, i may instead be treated as a hyperparameter. The value of i that results in the lowest loss value 62 during training may be selected in such examples by cross-validating the simulated dynamics of the CG structure 30.

At the molecular dynamics simulation module 80, the processor 12 may be configured to generate the molecular dynamics simulation 86 at least in part by approximating a solution to a Langevin equation 82 that includes the CG force field estimate 42. Using the denoising force field F_{z}^{DFF}, CG molecular dynamics simulations 86 may be computed by propagating the following Langevin equation 82 at the numerical solver 84:

$$M \frac{{d}^{2} z}{{dt}^{2}} = - {∇}_{z} V ( z ) - γ M \frac{dz}{dt} + \sqrt{2 M γ {k}_{B}} w ( t )$$

In the Langevin equation 82 as shown above, the substitution −∇₂V(z)=K_{B} [symbol] F_{z}^{DFF} is performed. In addition, M is a diagonal matrix M=diag(m₁, . . . , mₙ) that includes the masses of the CG beads 31, γ is a friction coefficient, and w(t) is a delta-correlated stationary Gaussian process [symbol] ₚ₍ₓ₎[w(t)·w(t′)]=δ(t−t′) with mean [symbol] ₚ₍ₓ₎[w(t)]=0. The constants in the Langevin equation 82 may be set to the values of those constants used when generating the atomistic molecular dynamics simulation data 24. Therefore, given a trained noise prediction network Ee, the only remaining hyperparameter left to tune in such examples is the noise level i.

In one limit of the Langevin equation, the masses m₁, . . . , mₙ are negligible and the friction coefficient γ is large (with a finite η=γM). This limit is referred to as Brownian dynamics or overdamped Langevin dynamics. Iteratively diffusing and denoising for one step at the first noise level (i=1) of the denoising diffusion model is equivalent to running a Brownian dynamics or overdamped Langevin dynamics simulation with a timestep given by

$$dt \frac{M γ}{} = 1 - {\overline{α}}_{1} = {β}_{1} .$$

The noise prediction neural network 44 is now discussed in additional detail. The architecture of the noise prediction neural network ϵ_{θ} is chosen according to the physical symmetries of the CG structure 30. One such symmetry is that the CG force field estimate 52 is conservative, i.e., it is equal the negative gradient of a CG energy potential V_{θ}(z). Therefore, the noise prediction neural network ϵ_{θ}(zᵢ, i) is parameterized as the gradient of a neural network with a scalar output: ϵ_{θ}(zᵢ, i)=∇_{z}_{ᵢ}nn_{θ}(z, i) with nn_{θ}: [symbol] ³ⁿ×{1, . . . , L} [symbol] [symbol] . The score ∇_{z}_{ᵢ} log q(zᵢ) that is used in the expression for the loss function 60 is parameterized as the gradient of an energy function. Using a conservative score function at the denoising diffusion model 40 allows stable molecular dynamics simulations 86 to be computed using the extracted CG force field estimate 42.

The noise prediction neural network 44 also exhibits translation invariance and rotation equivariance. Translation invariance holds ϵ_{θ}(z)=ϵ_{θ}(z+t) with t∈ [symbol] ³. This symmetry may be imposed when specifying the runtime conformer 70 by inputting the coordinates of the CG beads 31 into the noise prediction neural network 44 in the form of a plurality of pairwise difference vectors 72. Each of the pairwise difference vectors 72 may have the form z₍ᵢ₎-z₍ⱼ₎, where i and j are indices of the CG beads 31. Accordingly, the pairwise difference vectors 72 may indicate distances between the CG beads 31 in the runtime conformer 70. The noise prediction neural network 44 may, for example, be a two-layer graph transformer adapted with the above symmetry constraints. Since the noise prediction neural network 44 is translation-invariant and rotation-equivariant, the CG force field estimate 52 may also be translation-invariant and rotation-equivariant.

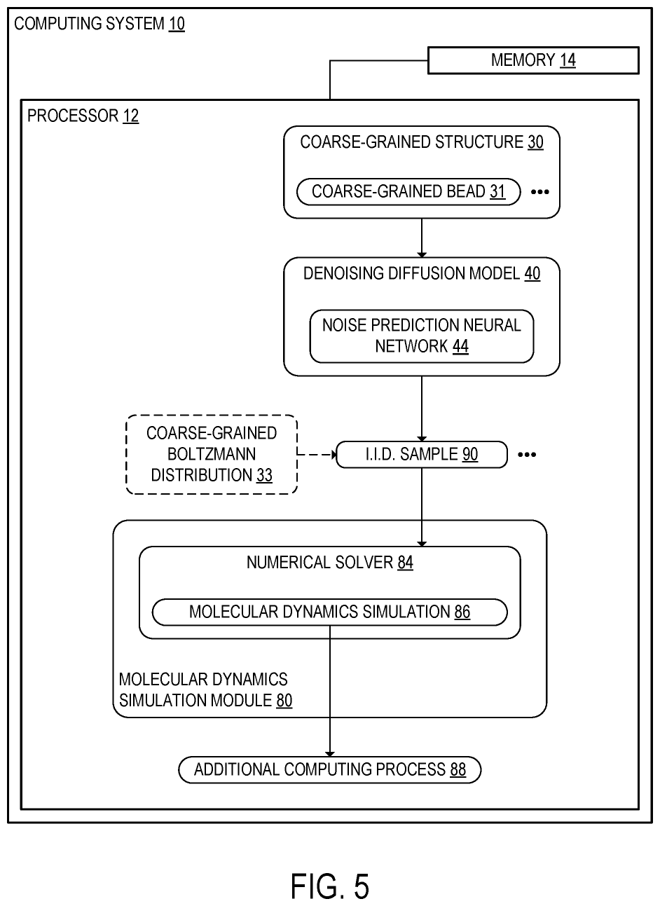

FIG. 5 schematically shows the computing system 10 in an example in which the processor 12 is further configured to generate a plurality of independent identically distributed (i.i.d.) samples 90 at the denoising diffusion model 40. The i.i.d. samples 90 may be sampled from an equilibrium distribution of the CG structure 30, which is the CG Boltzmann distribution 33 in the example of FIG. 5. Such i.i.d. samples 90 may be computed without explicitly generating the CG Boltzmann distribution 33. As discussed above with reference to FIGS. 1 and 3, the CG molecular dynamics simulation data 34 used to train the denoising diffusion model 40 may be a set of equilibrium samples from the CG Boltzmann distribution

$$q ( {z}_{0} ) = p ( z ) ∝ {e}^{- \frac{V ( z )}{}} .$$

Using the trained denoising diffusion model 40 parameterized with the noise prediction neural network 44, the i.i.d. samples 90 of the approximate CG equilibrium distribution may be generated through ancestral sampling from the graphical model p(z_{L})Πᵢ₌₁^{L}p_{θ}(zᵢ₋₁|zᵢ).

The processor 12 may be further configured to output the plurality of i.i.d. samples 90 to the molecular dynamics simulation module 80. At the molecular dynamics simulation module 80, the processor 12 may be further configured to generate the molecular dynamics simulation 86 of the molecule based at least in part on the plurality of i.i.d. samples 90.

Experimental results are discussed below for examples in which alanine dipeptide and the fast-folding proteins shown in FIG. 2 were simulated. In these experimental results, the denoising diffusion model 40 (which is referred to as DFF (sim.) and DFF (i.i.d.) when used for force field estimation and i.i.d. sample generation, respectively) is compared to three different baselines: CGNet sim., Flow i.i.d., and Flow-CGNet sim. CGNet sim. is a pure force-matching neural network trained on CG forces that were sliced from the fine-grained representation. Flow i.i.d. is a force-agnostic normalizing flow trained on the CG molecular dynamics simulation data 34. Flow-CGNet sim. is a force field obtained with a teacher-student setup by training a force-matching neural network (CGNet, “student”) on a gradient of a flow model (“teacher”). The denoising diffusion model 40 discussed above does not require a teacher-student setup, since the symmetry constraints are already integrated into the network of the denoising diffusion model 40. Thus, the denoising diffusion model 40 may be used as both a force field simulator and an i.i.d. sampler without further training. The results obtained from each of the above models may be compared to ground truth reference data obtained from a fine-grained molecular dynamics simulation.

The denoising diffusion model 40 was first evaluated on the CG structure 30A of alanine dipeptide for an all-atom simulation. The CG structure 30A, as shown in FIG. 2, slices out the five central backbone atoms of the molecule as the CG beads 31. The CG molecular dynamics simulation data 34 was generated over four independent runs, each of length 500 ns in simulated time, with 250,000 sample points saved per simulation (2 ps interval). The model was evaluated in a four-fold cross-validation setup, where three of the simulations were used for training-validation and one was used for testing.

In the Langevin dynamics simulation of the alanine dipeptide molecule, the system was simulated for 1 million iterations with a timestep resolution of 2 femtoseconds. Samples were saved every 250 iterations, resulting in 4K samples per simulation. 100 simulations were run in parallel, resulting in 400,000 total samples. The mass of each coarse-grained node was set to 12.8 g/mol, the weighted average of the mass of the carbon and oxygen atoms in the molecule. The temperature and the friction coefficient were set to 300 K and 1 ps⁻¹, respectively.

The noise prediction neural network 44 used in the alanine dipeptide simulations was a 2-layer graph transformer with 96 hidden features per layer. The neural network was trained with a learning rate of 3·10⁻⁴ and a cosine learning rate decay dropping to 1·10⁻⁵. The denoising diffusion model 40 was evaluated after training on the following amounts of training data: {10K, 20K, 50K, 100K, 200K, 500K}. The batch size was 1024. Early stopping was performed for the training set sizes 10K and 20K. The following values of the noise level i were obtained via cross-validation for different numbers of samples:

| | Training samples | | | | | |
|---|---|---|---|---|---|---|
| | 10K | 20K | 50K | 100K | 200K | 500K |
| Noise level i | 26 | 25 | 20 | 19 | 17 | 8 |

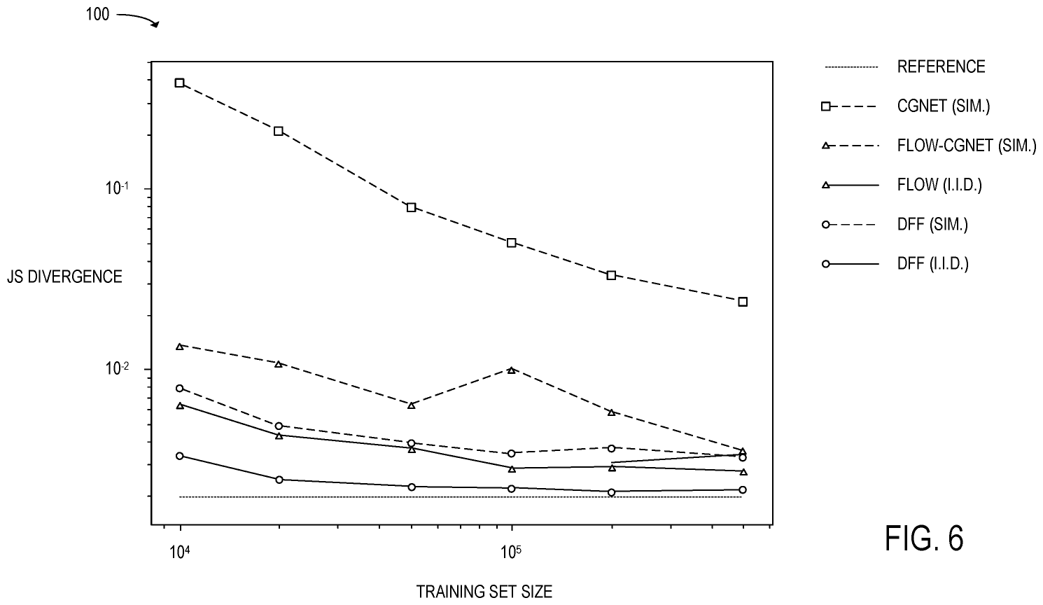

In the alanine dipeptide simulations, two dihedral angles ϕ and ψ along the coarse-grained backbone of the alanine dipeptide molecule were computed. Both of these angles are governed by four-body interactions that describe the main degrees of freedom of the CG structure 30A. FIG. 6 shows a plot 100 of Jensen-Shannon (JS) divergence between joint distributions of the dihedral angles ϕ and ψ in the training set and the validation partition when the CG structure 30A is modeled using different techniques. The JS divergence is shown for CGNet (sim.), Flow-CGNet (sim.), Flow (i.i.d.), Reference, DFF (sim.), and DFF (i.i.d.) after the different neural networks were each trained with 500K samples.

As shown in FIG. 6, DFF sim. outperforms previous CG simulation methods (Flow-CGNet sim. and CGNet sim.) in all tested data regimes. In the low data regime, DFF is more data efficient than previous methods in both i.i.d. and simulation settings. DFF sim. also outperforms previous models in larger data regimes, closely matching the JS Divergence of the lower bound.

Experiments were also performed in which respective denoising diffusion models 40 utilizing the denoising force field techniques discussed above were trained for five fast-folding proteins: chignolin, Trp-cage, Bba, villin, and protein G. The fine-grained and coarse-grained structures of these proteins are shown in FIG. 2, as discussed above. The simulations varied in length, but for each trajectory, the frames were randomly shuffled and split 70-10-20% into a training, validation, and test set. The interval between frames was 200 picoseconds for each of the proteins. In the denoising-diffusion process, a cosine scheduler with 1000 different noise levels i was used.

The following table shows additional details related to the training of the denoising diffusion models 40 for the proteins:

| | Chignolin | Trp-cage | Bba | Villin | Protein G |
|---|---|---|---|---|---|
| ID | CLN025 | 2JOF | 1FME | 2F4K | NuG2/ |
| | | | | | 1MIO |
| Temperature | 340 | 290 | 325 | 360 | 350 |
| (K) | | | | | |
| Amino acids | 10 | 20 | 28 | 35 | 56 |
| Simulation | 106 | 208 | 223 | 125 | 369 |
| length (μs) | | | | | |
| Data points | 534,743 | 1,044,000 | 1,114,545 | 629,907 | 1,849,251 |

The following table shows hyperparameters used at the denoising diffusion models 40 in the protein modeling experiments:

| | Chignolin | Trp-cage | Bba | Villin | Protein G |
|---|---|---|---|---|---|
| Batch size | 512 | 512 | 512 | 512 | 256 |
| Learning | 4 · 10⁻⁴ | 4 · 10⁻⁴ | 4 · 10⁻⁴ | 4 · 10⁻⁴ | 4 · 10⁻⁴ |
| rate | | | | | |
| Training | 1M | 1M | 2M | 2M | 3M |
| iterations | | | | | |
| Layers | 3 | 3 | 3 | 3 | 3 |
| Features | 64 | 128 | 96 | 128 | 128 |
| Exponential | .995 | .995 | .995 | .995 | 3 |
| moving | | | | | |
| average | | | | | |

In the Langevin dynamics simulation, 6 million simulation steps were performed, and samples were saved every 500 steps. 100 simulations were run in parallel, resulting in 1.2M samples. The masses of the particles were set to 12 g/mol. The temperatures used in the Langevin dynamics simulation were the same temperatures used in the ground truth data.

The following table shows the JS divergence of time-lagged independent component (TIC) distributions and pairwise distance (PWD) distributions for each of the fast-folding proteins. In the following table, the denoising diffusion model is compared to Flow for the i.i.d. sample generation task and to Flow-CGNet for the force field simulation task.

| | Chignolin | | Trp-cage | | Bba | | Villin | | Protein G | |
|---|---|---|---|---|---|---|---|---|---|---|
| | TIC JS | PWD JS | TIC JS | PWD JS | TIC JS | PWD JS | TIC JS | PWD JS | TIC JS | PWD JS |
| Ref. | .0057 | .0002 | .0026 | .0002 | .0040 | .0002 | .0032 | .0004 | .0014 | .0002 |
| Flow (i.i.d.) | .0106 | .0022 | .0078 | .0057 | .0229 | .0073 | .0109 | .0142 | n/a | n/a |
| DFF (i.i.d.) | .0096 | .0005 | .0052 | .0007 | .0111 | .0017 | .0073 | .0009 | .0131 | .0009 |
| Flow-CGNet (sim.) | .1875 | .1271 | .1009 | .0474 | .1469 | .0594 | .2153 | .0535 | n/a | n/a |
| DFF (sim.) | .0335 | .0067 | .0518 | .0403 | .1289 | .0408 | .0564 | .0244 | .2260 | .0691 |

As shown in the above table, DFF (i.i.d.) and DFF (sim.) both outperform the baseline approaches Flow (i.i.d.) and Flow-CGNet (sim.) on both equilibrium metrics TIC JS and PWD JS.

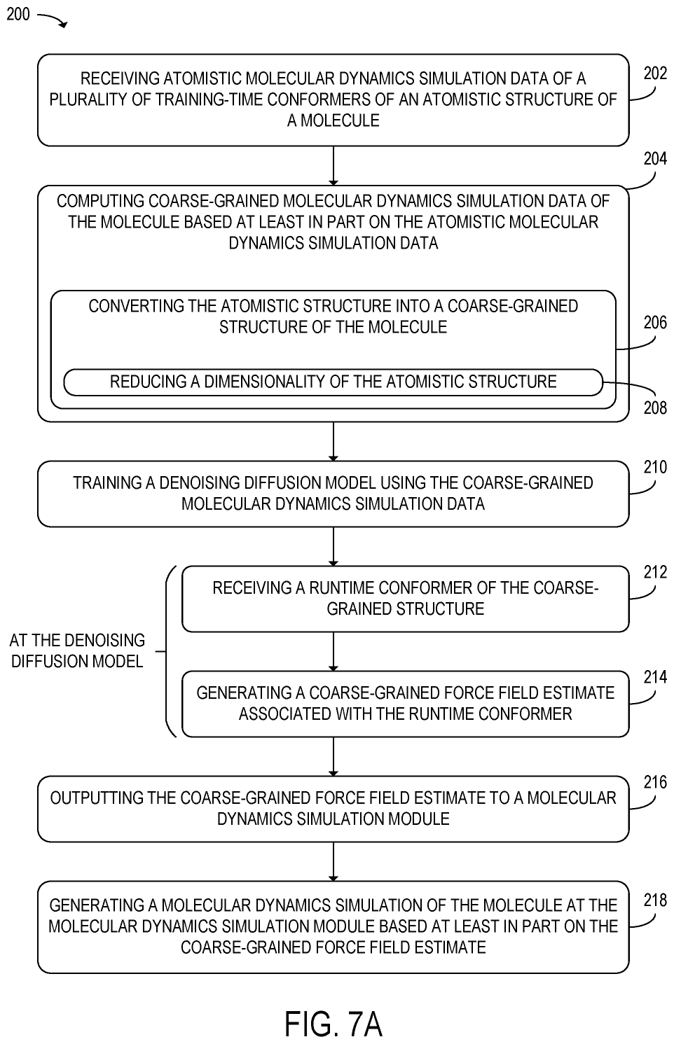

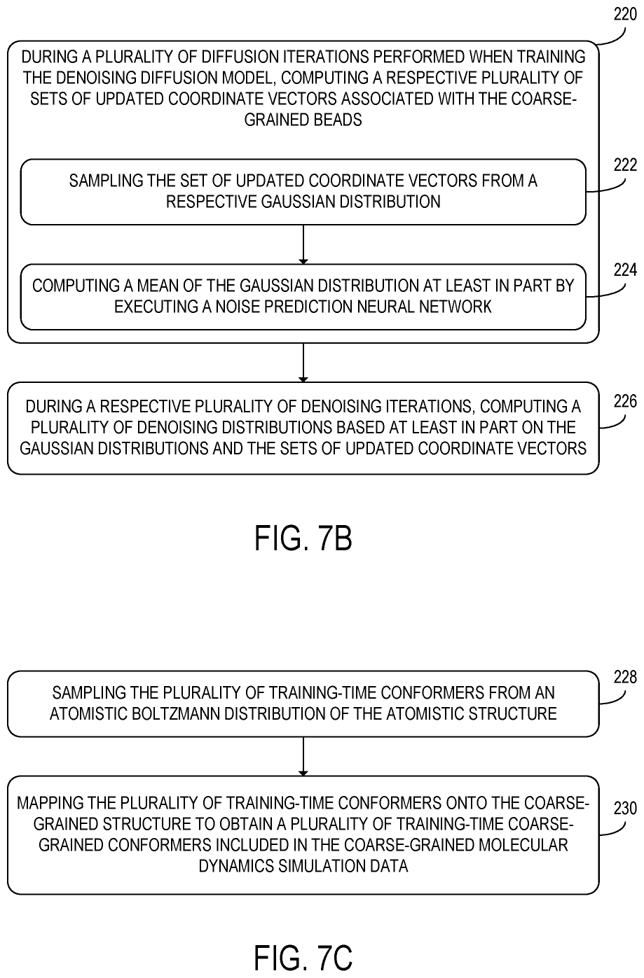

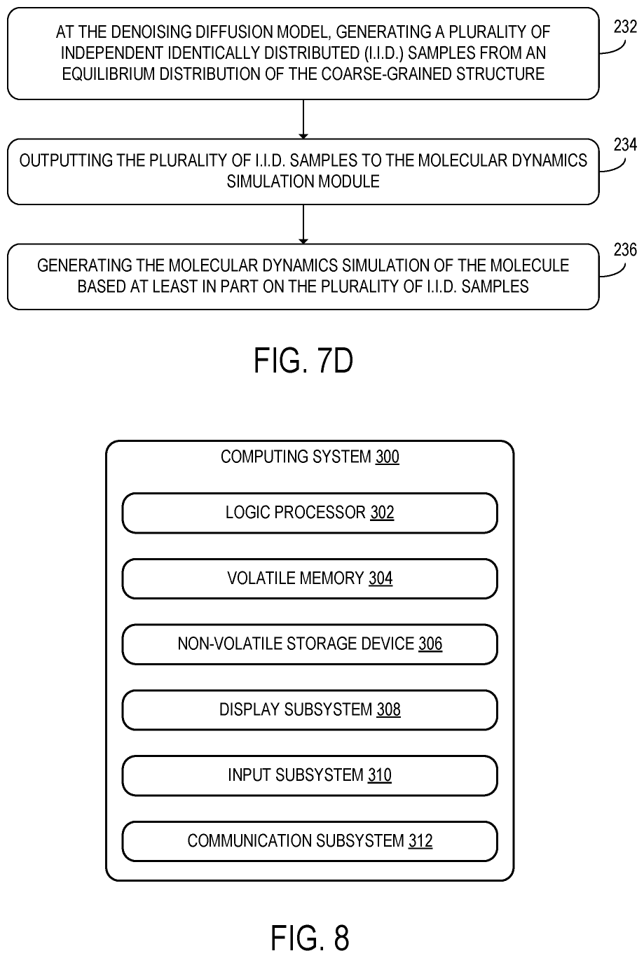

FIG. 7A shows a flowchart of method 200 for use with a computing system to perform training and inferencing at a denoising diffusion model used in molecular dynamics simulation. At step 202, the method 200 includes receiving atomistic molecular dynamics simulation data of a plurality of training-time conformers of an atomistic structure of a molecule. The training-time conformers may be associated with respective training simulation timesteps. Accordingly, atomistic molecular dynamics simulation data may include sets of training-time conformers that provide examples of movement trajectories over time for the atoms included in the atomistic structure.

At step 204, the method 200 further includes computing CG molecular dynamics simulation data of the molecule based at least in part on the atomistic molecular dynamics simulation data. Step 204 may include, at step 206, converting the atomistic structure into a coarse-grained structure of the molecule when computing the coarse-grained molecular dynamics simulation data. In addition, at step 208, generating the coarse-grained structure at step 206 may include reducing a dimensionality of the atomistic structure. A coarse-graining map that projects the atoms of the atomistic structure onto a plurality of coarse-grained beads included in the CG structure may be used to reduce the dimensionality of the atomistic structure.

At step 210, the method 200 further includes training a denoising diffusion model using the coarse-grained molecular dynamics simulation data. The denoising diffusion model may be trained using a loss function that includes terms proportional to a gradient of an energy function. The loss function may result in a denoising diffusion network that models conservative forces exerted by the molecule.

Steps 212 and 214 are performed at the denoising diffusion model during runtime. At step 212, the method 200 further includes receiving a runtime conformer of the CG structure. At step 214, the method 200 further includes generating a CG force field estimate associated with the runtime conformer. In some examples, the runtime conformer may be specified by a plurality of pairwise difference vectors that indicate distances between the CG beads. In such examples, the CG force field estimate may be translation-invariant and rotation-equivariant.

At step 216, the method 200 further includes outputting the CG force field estimate to a molecular dynamics simulation module. At step 218, the method 200 further includes generating a molecular dynamics simulation of the molecule at the molecular dynamics simulation module based at least in part on the CG force field estimate. For example, generating the molecular dynamics simulation may include numerically approximating a solution to a Langevin equation that includes the coarse-grained force field estimate. In such examples, the Langevin equation may be used to model Brownian motion of the molecule.

FIG. 7B shows additional steps of the method 200 that may be performed in some examples when training the denoising diffusion model. At step 220, during a plurality of diffusion iterations performed when training the denoising diffusion model, the method 200 may further include computing a respective plurality of sets of updated coordinate vectors associated with the coarse-grained beads. Step 220 may include, at step 222, sampling the set of updated coordinate vectors from a respective Gaussian distribution. Accordingly, Gaussian noise may be applied to the training-time conformers during the diffusion phase of training.

Computing the updated coordinate vectors at step 220 may further include, at step 224, computing a mean of the Gaussian distribution at least in part by executing a noise prediction neural network. The noise prediction neural network may be a graph transformer network in some examples. The mean may be used to parameterize the Gaussian distribution from which the updated coordinate vectors are sampled during the diffusion iteration.

At step 226, the method 200 may further include computing a plurality of denoising distributions during a respective plurality of denoising iterations. The denoising distributions may be computed based at least in part on the Gaussian distributions and the sets of updated coordinate vectors. The Gaussian distributions may be parameterized using the means computed in the diffusion iterations at step 224. During the denoising iterations, the denoising diffusion network may learn an inverse of the diffusion process.

FIG. 7C shows additional steps of the method 200 that may be performed when the CG molecular dynamics simulation data is computed at step 204. At step 228, the method 200 may further include sampling the plurality of training-time conformers from an atomistic Boltzmann distribution of the atomistic structure. At step 230, the method 200 may further include mapping the plurality of training-time conformers onto the coarse-grained structure to obtain a plurality of training-time coarse-grained conformers included in the coarse-grained molecular dynamics simulation data. Thus, the training-time conformers sampled from the atomistic Boltzmann distribution may be converted into CG conformers sampled from a CG Boltzmann distribution.

FIG. 7D shows steps of the method 200 that may be performed additionally or alternatively to generating and outputting the CG force field estimate. At step 232, the method 200 may further include generating, at the denoising diffusion model, a plurality of i.i.d. samples from an equilibrium distribution of the coarse-grained structure. The method 200 may further include, at step 234, outputting the plurality of i.i.d. samples to the molecular dynamics simulation module. At step, 236, the method 200 may further include generating the molecular dynamics simulation of the molecule at the molecular dynamics simulation module based at least in part on the plurality of i.i.d. samples. Accordingly, the denoising diffusion network may be used to generate i.i.d. samples as well as force field estimates.

In some embodiments, the methods and processes described herein may be tied to a computing system of one or more computing devices. In particular, such methods and processes may be implemented as a computer-application program or service, an application-programming interface (API), a library, and/or other computer-program product.

FIG. 3 schematically shows a non-limiting embodiment of a computing system 300 that can enact one or more of the methods and processes described above. Computing system 300 is shown in simplified form. Computing system 300 may embody the computing system 10 described above and illustrated in FIG. 1. Components of computing system 300 may be instantiated in one or more personal computers, server computers, tablet computers, home-entertainment computers, network computing devices, video game devices, mobile computing devices, mobile communication devices (e.g., smart phone), and/or other computing devices, and wearable computing devices such as smart wristwatches and head mounted augmented reality devices.

Computing system 300 includes a logic processor 302 volatile memory 304, and a non-volatile storage device 306. Computing system 300 may optionally include a display subsystem 308, input subsystem 310, communication subsystem 312, and/or other components not shown in FIG. 8.

Logic processor 302 includes one or more physical devices configured to execute instructions. For example, the logic processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors configured to execute software instructions. Additionally or alternatively, the logic processor may include one or more hardware logic circuits or firmware devices configured to execute hardware-implemented logic or firmware instructions. Processors of the logic processor 302 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor may be virtualized and executed by remotely accessible, networked computing devices configured in a cloud-computing configuration. In such a case, these virtualized aspects are run on different physical logic processors of various different machines, it will be understood.

Non-volatile storage device 306 includes one or more physical devices configured to hold instructions executable by the logic processors to implement the methods and processes described herein. When such methods and processes are implemented, the state of non-volatile storage device 306 may be transformed—e.g., to hold different data.

Non-volatile storage device 306 may include physical devices that are removable and/or built-in. Non-volatile storage device 306 may include optical memory, semiconductor memory, and/or magnetic memory, or other mass storage device technology. Non-volatile storage device 306 may include nonvolatile, dynamic, static, read/write, read-only, sequential-access, location-addressable, file-addressable, and/or content-addressable devices. It will be appreciated that non-volatile storage device 306 is configured to hold instructions even when power is cut to the non-volatile storage device 306.

Volatile memory 304 may include physical devices that include random access memory. Volatile memory 304 is typically utilized by logic processor 302 to temporarily store information during processing of software instructions. It will be appreciated that volatile memory 304 typically does not continue to store instructions when power is cut to the volatile memory 304.

Aspects of logic processor 302, volatile memory 304, and non-volatile storage device 306 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 300 typically implemented in software by a processor to perform a particular function using portions of volatile memory, which function involves transformative processing that specially configures the processor to perform the function. Thus, a module, program, or engine may be instantiated via logic processor 302 executing instructions held by non-volatile storage device 306, using portions of volatile memory 304. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 308 may be used to present a visual representation of data held by non-volatile storage device 306. The visual representation may take the form of a graphical user interface (GUI). As the herein described methods and processes change the data held by the non-volatile storage device, and thus transform the state of the non-volatile storage device, the state of display subsystem 308 may likewise be transformed to visually represent changes in the underlying data. Display subsystem 308 may include one or more display devices utilizing virtually any type of technology. Such display devices may be combined with logic processor 302, volatile memory 304, and/or non-volatile storage device 306 in a shared enclosure, or such display devices may be peripheral display devices.

When included, input subsystem 310 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor.

When included, communication subsystem 312 may be configured to communicatively couple various computing devices described herein with each other, and with other devices. Communication subsystem 312 may include wired and/or wireless communication devices compatible with one or more different communication protocols. As non-limiting examples, the communication subsystem may be configured for communication via a wireless telephone network, or a wired or wireless local- or wide-area network. In some embodiments, the communication subsystem may allow computing system 300 to send and/or receive messages to and/or from other devices via a network such as the Internet.

The following paragraphs discuss several aspects of the present disclosure. According to one aspect of the present disclosure, a computing system is provided, including a processor configured to receive atomistic molecular dynamics simulation data of a plurality of training-time conformers of an atomistic structure of a molecule. The processor is further configured to compute coarse-grained molecular dynamics simulation data of the molecule based at least in part on the atomistic molecular dynamics simulation data. The coarse-grained molecular dynamics simulation data is computed at least in part by converting the atomistic structure into a coarse-grained structure of the molecule. The processor is further configured to train a denoising diffusion model using the coarse-grained molecular dynamics simulation data. At the denoising diffusion model, the processor is further configured to receive a runtime conformer of the coarse-grained structure and generate a coarse-grained force field estimate associated with the runtime conformer. The processor is further configured to output the coarse-grained force field estimate to a molecular dynamics simulation module. The processor is further configured to generate a molecular dynamics simulation of the molecule at the molecular dynamics simulation module based at least in part on the coarse-grained force field estimate. The above features may have the technical effect of accurately simulating molecular dynamics in a manner that scales to larger molecule sizes more easily than previous approaches.

According to this aspect, the processor may be configured to generate the coarse-grained structure at least in part by reducing a dimensionality of the atomistic structure. The above feature may have the technical effect of reducing the number of variables the denoising diffusion model estimates, thereby allowing for more efficient training and inferencing.

According to this aspect, the coarse-grained structure may include a plurality of coarse-grained beads. The above feature may have the technical effect of reducing the dimensionality of the atomistic structure by grouping together sets of atoms that move together.

According to this aspect, during a plurality of diffusion iterations performed when training the denoising diffusion model, the processor may be configured to compute a respective plurality of sets of updated coordinate vectors associated with the coarse-grained beads. The above features may have the technical effect of applying noise to the coordinate vectors of the coarse-grained beads during a diffusion phase of training.

According to this aspect, the processor may be configured to perform each of the diffusion iterations at least in part by sampling the set of updated coordinate vectors from a respective Gaussian distribution. The above features may have the technical effect of randomly or pseudorandomly generating the noise applied to the coordinate vectors during the diffusion phase.

According to this aspect, during each of the diffusion iterations, the processor may be further configured to compute a mean of the Gaussian distribution at least in part by executing a noise prediction neural network. The above features may have the technical effect of determining the amounts of noise applied to the coordinate vectors.

According to this aspect, the noise prediction neural network is a graph transformer network. The above feature may have the technical effect of using a noise prediction neural network architecture that accurately and efficiently models the noise applied to the CG structure.

According to this aspect, the processor is configured to compute the coarse-grained molecular dynamics simulation data at least in part by sampling the plurality of training-time conformers from an atomistic Boltzmann distribution of the atomistic structure. Computing the coarse-grained molecular dynamics simulation data may further include mapping the plurality of training-time conformers onto the coarse-grained structure to obtain a plurality of training-time coarse-grained conformers included in the coarse-grained molecular dynamics simulation data. The above features may have the technical effect of compressing the atomistic molecular dynamics simulation data into a more quickly learnable form.

According to this aspect, at the molecular dynamics simulation module, the processor may be configured to generate the molecular dynamics simulation at least in part by approximating a solution to a Langevin equation that includes the coarse-grained force field estimate. The above features may have the technical effect of simulating the motion of the molecule from the CG force field estimate.

According to this aspect, the coarse-grained force field estimate may be translation-invariant and rotation-equivariant. The above features may have the technical effect of reflecting physical symmetries of the CG structure in the CG force field estimate.

According to this aspect, the runtime conformer may be specified by a plurality of pairwise difference vectors. The above feature may have the technical effect of encoding the translation invariance and the rotation equivariance in the structure of the input data of the denoising diffusion model.

According to this aspect, the processor may be further configured to generate a plurality of independent identically distributed (i.i.d.) samples from an equilibrium distribution of the coarse-grained structure at the denoising diffusion model. The processor may be further configured to output the plurality of i.i.d. samples to the molecular dynamics simulation module. The processor may be further configured to generate the molecular dynamics simulation of the molecule based at least in part on the plurality of i.i.d. samples. The above features may have the technical effect of generating i.i.d. samples of the CG structure at the same machine learning model used for generating the CG force field estimates.

According to another aspect of the present disclosure, a method for use with a computing system is provided. The method includes receiving atomistic molecular dynamics simulation data of a plurality of training-time conformers of an atomistic structure of a molecule. The method further includes computing coarse-grained molecular dynamics simulation data of the molecule based at least in part on the atomistic molecular dynamics simulation data. The coarse-grained molecular dynamics simulation data is computed at least in part by converting the atomistic structure into a coarse-grained structure of the molecule. The method further includes training a denoising diffusion model using the coarse-grained molecular dynamics simulation data. At the denoising diffusion model, the method further includes receiving a runtime conformer of the coarse-grained structure and generating a coarse-grained force field estimate associated with the runtime conformer. The method further includes outputting the coarse-grained force field estimate to a molecular dynamics simulation module. The method further includes generating a molecular dynamics simulation of the molecule at the molecular dynamics simulation module based at least in part on the coarse-grained force field estimate. The above features may have the technical effect of accurately simulating molecular dynamics in a manner that scales to larger molecule sizes more easily than previous approaches.

According to this aspect, generating the coarse-grained structure may include reducing a dimensionality of the atomistic structure. The coarse-grained structure includes a plurality of coarse-grained beads. The above features may have the technical effect of reducing the number of variables the denoising diffusion model estimates by grouping together sets of atoms that move together, thereby allowing for more efficient training and inferencing.

According to this aspect, during a plurality of diffusion iterations performed when training the denoising diffusion model, the method may further include computing a respective plurality of sets of updated coordinate vectors associated with the coarse-grained beads. The above features may have the technical effect of applying noise to the coordinate vectors of the coarse-grained beads during a diffusion phase of training.

According to this aspect, performing each of the diffusion iterations may further include sampling the set of updated coordinate vectors from a respective Gaussian distribution. Performing each of the diffusion iterations may further include computing a mean of the Gaussian distribution at least in part by executing a noise prediction neural network. The above features may have the technical effect of randomly or pseudorandomly generating the noise applied to the coordinate vectors during the diffusion phase. The above features may have the further technical effect of determining the amounts of noise applied to the coordinate vectors.

According to this aspect, computing the coarse-grained molecular dynamics simulation data may include sampling the plurality of training-time conformers from an atomistic Boltzmann distribution of the atomistic structure. Computing the coarse-grained molecular dynamics simulation data may further include mapping the plurality of training-time conformers onto the coarse-grained structure to obtain a plurality of training-time coarse-grained conformers included in the coarse-grained molecular dynamics simulation data. The above features may have the technical effect of compressing the atomistic molecular dynamics simulation data into a more quickly learnable form.

According to this aspect, the coarse-grained force field estimate may be translation-invariant and rotation-equivariant. The above features may have the technical effect of reflecting physical symmetries of the CG structure in the CG force field estimate.

According to this aspect, the method may further include, at the denoising diffusion model, generating a plurality of independent identically distributed (i.i.d.) samples from an equilibrium distribution of the coarse-grained structure. The method may further include outputting the plurality of i.i.d. samples to the molecular dynamics simulation module. The method may further include generating the molecular dynamics simulation of the molecule based at least in part on the plurality of i.i.d. samples. The above features may have the technical effect of generating i.i.d. samples of the CG structure at the same machine learning model used for generating the CG force field estimates.

According to another aspect of the present disclosure, a computing system is provided, including a processor configured to receive atomistic molecular dynamics simulation data of a plurality of training-time conformers of an atomistic structure of a molecule. The processor is further configured to compute coarse-grained molecular dynamics simulation data of the molecule based at least in part on the atomistic molecular dynamics simulation data. The coarse-grained molecular dynamics simulation data may be computed at least in part by converting the atomistic structure into a coarse-grained structure of the molecule. The processor is further configured to train a denoising diffusion model using the coarse-grained molecular dynamics simulation data. At the denoising diffusion model, the processor is further configured to generate a plurality of independent identically distributed (i.i.d.) samples from an equilibrium distribution of the coarse-grained structure. The processor is further configured to output the plurality of i.i.d. samples. The above features may have the technical effect of efficiently performing training and inferencing at a denoising diffusion model to generate i.i.d. samples of a distribution of conformers of a molecule.

“And/or” as used herein is defined as the inclusive or V, as specified by the following truth table:

| A | B | A ∨ B |
|---|---|---|
| True | True | True |
| True | False | True |
| False | True | True |
| False | False | False |

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system comprising:
a processor configured to:
receive atomistic molecular dynamics simulation data of a plurality of training-time conformers of an atomistic structure of a molecule;
compute coarse-grained molecular dynamics simulation data of the molecule based at least in part on the atomistic molecular dynamics simulation data, wherein the coarse-grained molecular dynamics simulation data is computed at least in part by converting the atomistic structure into a coarse-grained structure of the molecule;
train a denoising diffusion model using the coarse-grained molecular dynamics simulation data;
at the denoising diffusion model:
receive a runtime conformer of the coarse-grained structure; and
generate a coarse-grained force field estimate associated with the runtime conformer;
output the coarse-grained force field estimate to a molecular dynamics simulation module; and
generate a molecular dynamics simulation of the molecule at the molecular dynamics simulation module based at least in part on the coarse-grained force field estimate.

2. The computing system of claim 1, wherein the processor is configured to generate the coarse-grained structure at least in part by reducing a dimensionality of the atomistic structure.

3. The computing system of claim 2, wherein the coarse-grained structure includes a plurality of coarse-grained beads.

4. The computing system of claim 3, wherein, during a plurality of diffusion iterations performed when training the denoising diffusion model, the processor is configured to compute a respective plurality of sets of updated coordinate vectors associated with the coarse-grained beads.

5. The computing system of claim 4, wherein the processor is configured to perform each of the diffusion iterations at least in part by sampling the set of updated coordinate vectors from a respective Gaussian distribution.

6. The computing system of claim 5, wherein, during each of the diffusion iterations, the processor is further configured to compute a mean of the Gaussian distribution at least in part by executing a noise prediction neural network.

7. The computing system of claim 6, wherein the noise prediction neural network is a graph transformer network.

8. The computing system of claim 3, wherein the processor is configured to compute the coarse-grained molecular dynamics simulation data at least in part by:
sampling the plurality of training-time conformers from an atomistic Boltzmann distribution of the atomistic structure; and
mapping the plurality of training-time conformers onto the coarse-grained structure to obtain a plurality of training-time coarse-grained conformers included in the coarse-grained molecular dynamics simulation data.

9. The computing system of claim 1, wherein, at the molecular dynamics simulation module, the processor is configured to generate the molecular dynamics simulation at least in part by approximating a solution to a Langevin equation that includes the coarse-grained force field estimate.

10. The computing system of claim 1, wherein the coarse-grained force field estimate is translation-invariant and rotation-equivariant.

11. The computing system of claim 10, wherein the runtime conformer is specified by a plurality of pairwise difference vectors.

12. The computing system of claim 1, wherein the processor is further configured to:
at the denoising diffusion model, generate a plurality of independent identically distributed (i.i.d.) samples from an equilibrium distribution of the coarse-grained structure;
output the plurality of i.i.d. samples to the molecular dynamics simulation module; and
generate the molecular dynamics simulation of the molecule based at least in part on the plurality of i.i.d. samples.

13. A method for use with a computing system, the method comprising;
receiving atomistic molecular dynamics simulation data of a plurality of training-time conformers of an atomistic structure of a molecule;
computing coarse-grained molecular dynamics simulation data of the molecule based at least in part on the atomistic molecular dynamics simulation data, wherein the coarse-grained molecular dynamics simulation data is computed at least in part by converting the atomistic structure into a coarse-grained structure of the molecule;
training a denoising diffusion model using the coarse-grained molecular dynamics simulation data;
at the denoising diffusion model:
receiving a runtime conformer of the coarse-grained structure; and
generating a coarse-grained force field estimate associated with the runtime conformer;
outputting the coarse-grained force field estimate to a molecular dynamics simulation module; and
generating a molecular dynamics simulation of the molecule at the molecular dynamics simulation module based at least in part on the coarse-grained force field estimate.

14. The method of claim 13, wherein:
generating the coarse-grained structure includes reducing a dimensionality of the atomistic structure; and
the coarse-grained structure includes a plurality of coarse-grained beads.

15. The method of claim 14, further comprising, during a plurality of diffusion iterations performed when training the denoising diffusion model, computing a respective plurality of sets of updated coordinate vectors associated with the coarse-grained beads.

16. The method of claim 15, wherein performing each of the diffusion iterations further includes:
sampling the set of updated coordinate vectors from a respective Gaussian distribution; and
computing a mean of the Gaussian distribution at least in part by executing a noise prediction neural network.

17. The method of claim 14, wherein computing the coarse-grained molecular dynamics simulation data includes:
sampling the plurality of training-time conformers from an atomistic Boltzmann distribution of the atomistic structure; and
mapping the plurality of training-time conformers onto the coarse-grained structure to obtain a plurality of training-time coarse-grained conformers included in the coarse-grained molecular dynamics simulation data.

18. The method of claim 13, wherein the coarse-grained force field estimate is translation-invariant and rotation-equivariant.

19. The method of claim 13, further comprising:
at the denoising diffusion model, generating a plurality of independent identically distributed (i.i.d.) samples from an equilibrium distribution of the coarse-grained structure;
outputting the plurality of i.i.d. samples to the molecular dynamics simulation module; and
generating the molecular dynamics simulation of the molecule based at least in part on the plurality of i.i.d. samples.

20. A computing system comprising:
a processor configured to:
receive atomistic molecular dynamics simulation data of a plurality of training-time conformers of an atomistic structure of a molecule;
compute coarse-grained molecular dynamics simulation data of the molecule based at least in part on the atomistic molecular dynamics simulation data, wherein the coarse-grained molecular dynamics simulation data is computed at least in part by converting the atomistic structure into a coarse-grained structure of the molecule;
train a denoising diffusion model using the coarse-grained molecular dynamics simulation data;
at the denoising diffusion model, generate a plurality of independent identically distributed (i.i.d.) samples from an equilibrium distribution of the coarse-grained structure; and
output the plurality of i.i.d. samples.
